(19) United States
(12) Patent Application Publication
(10) Pub. No.: US 2003/0086819 A1
(43) Pub. Date: May 8, 2003

(54) Pre-sterilized, sealed, disposable, pre-packaged, self-contained, reusable safety baby bottle assembly and feeding method

(76) Inventor: Donald J. Rea, Chino Valley, AZ (US)

Correspondence Address: Tod R. Nissle, Esq., TOD R. NISSLE, P.C., P.O. Box 55630, Phoenix, AZ, 85078, US

(21) Appl. No.: 10/108,271

(22) Filed: Mar. 26, 2002

Related U.S. Application Data
(60) Provisional application No. 60/336,902, filed on Nov. 7, 2001.

(51) Int. Cl. A01N 1/00; A61L 2/00

(52) U.S. Cl. 422/28; 422/32

(57) ABSTRACT

A method for a parent to safely administer formula to a baby. The method includes the step of providing a pre-packaged, pre-sterilized, self-contained, disposable safety bottle by sterilizing a container; sterilizing formula; charging the sterilized container with the sterilized formula; sealing the container; and, removably mounting a nipple assembly on the container. The nipple assembly includes a base, a nipple, and a removable cover extending over the nipple. The method also includes the steps of delivering the safety bottle to the parent; removing the nipple assembly from the container; unsealing the container; remounting the nipple assembly on the container; removing the cover; administering the formula to the baby; and, discarding the safety bottle.

This invention pertains to a method and apparatus for administering a liquid to a baby.

More particularly, the invention pertains to a method and apparatus to facilitate feeding a baby when the baby is traveling away from home.

In a further respect, the invention pertains to a method and apparatus for feeding a baby without requiring that formula be prepared by transferring formula from a storage container to a baby bottle.

In another respect, the invention pertains to a method and apparatus for reducing the risk that a baby is fed contaminated or disease laden formula or other liquids.

Most parents are unaware that it is unlikely that the formula they feed their babies is free of disease and contamination.

After the residents of a home go to bed, the quiet patter of little feet begins. Insects resident in homes throughout the country begin their travels. Spiders, cockroaches, ants and other insects travel over floors, up walls, through cupboards, over the tops of cans, over counter tops, and over food, baby nipples, baby bottles, and other items left on counter tops or in cupboards.

During its travels, an insect can leave a trail of disease and contamination.

Insecticides sprayed in homes kill many insects. Other insects are not killed, or are not killed immediately, and can leave traces of insecticide as the insect moves through a home.

Apart from insects, bacteria and viruses inhabit counter tops, utensils, dish towels, and other items. The existence of these bacteria is one reason anti-bacterial sprays and soaps are popular. The fact is, however, that bacteria are never completely eliminated. This is evidenced in hospitals, where cases of staph are common, despite the various commonly employed sterilization measures.

During the preparation of a bottle of formula for a baby, a can opener is utilized to open a can of liquid formula or of powdered formula. Since bacteria likely are present both on the top of the can and on the can opener, the act of opening the can introduces bacteria into the formula. The formula is then poured into a baby bottle. The baby bottle, or the disposable liner user in the baby bottle, likely has been sterilized. After the bottle was sterilized, it was placed on a dish towel or other surface. Since these surfaces contain bacteria, there is bacteria on the baby bottle. When the formula is poured through air into the baby bottle, the formula picks up contaminants in the air. The fact that the formula picks up contaminants in the air may seem insignificant. However, one concern associated with the injection with hypodermic needles of drugs into a human body is that ambient air is typically included in the hypodermic. The contaminants in the ambient air are believed possibly to adversely affect the medicine being introduced into the human body, or to affect the well-being of the individual being injected. The potential toxic affect of pollutants in the air is well documented.

By the time the formula is poured into a baby bottle, the formula likely is tainted with a variety of bacteria and contaminants, despite the well-intentioned efforts of the parent.

The baby nipple is then threaded onto the baby bottle. The baby bottle includes an externally threaded upper end. The nipple includes an internally threaded base. The nipple base is turned onto the externally threaded end of the baby bottle. The nipple is likely contaminated with bacteria. These bacteria or other contaminants can originate from the parent's hands, from a surface on which the nipple was resting, from insects, etc.

The completed baby bottle is then given to the baby, or, the parent holds the baby bottle while the infant suckles. If the completed baby bottle and the contents of the bottle were carefully examined with an electron microscope and other means, the multitude of bacteria and other contaminants present would probably astound and depress the parent.

Despite the fact that baby bottles carrying many contaminants are routinely administered to babies, the very large majority of babies survive, and apparently suffer no significant side effects. After all, each baby normally has a functioning immune system which protects the baby. The fact remains, however, that it is likely that in some instances bacteria or other contaminants contained on or in baby bottles cause injury or death to a baby, which death or injury may appear to an observer to have no discernable cause. Deadly bacteria and toxic contaminants exist that can, in very small quantities, cause serious injury to a baby, a child, or an adult.

While the common use of “sterilized” formula, baby bottles and bottle liners is useful, the use of these components in no way guarantees that a baby is fed formula that is truly sterile and free of bacteria and contaminants.

Another disadvantage of conventional procedures for feeding a baby is the number of separate steps required. Mixing and transferring formula is messy and requires a variety of implements including formula containers, nipples, baby bottles. Mothers have long coped with such problems and have successfully raised children for many years. A common and long standing complaint, however, of mothers are the many components required to feed a baby, especially when transporting the baby away from home. The size and numerous contents of baby bags are legion.

A further disadvantage of the conventional procedures for feeding babies is the likelihood that baby formula will leak or spill from storage containers or baby bottles, requiring cleaning of clothes, blankets, and bags.

Still another disadvantage of the foregoing conventional procedure for feeding babies is that during travel, a variety of implements has to be purchased at a grocery store in the event the feeding apparatus for the baby is lost or misplaced.

Accordingly, it would be highly desirable to provide an improved feeding method and apparatus which would significantly reduce the likelihood that the formula administered to a baby will include harmful bacteria or contaminants, would significantly reduce the number of separate steps required to administer formula to a baby, would reduce the risk of formula leakage, and would simplify the administration of formula to a baby during travel.

Therefore, it is a principal object of the instant invention to provide an improved method and apparatus for safely administering cow's milk, soy milk, or other formula to a baby.

Another object of the invention is to provide an improved safe feeding method and apparatus for significantly reducing the likelihood that the formula given a baby includes bacteria or other contaminants.

A further object of the invention is to provide an improved safe feeding method and apparatus that greatly simplifies the steps required to administer formula to a baby.

Still another object of the invention is to provide an improved safe feeding method and apparatus that simplifies the steps required to feed a baby during travel.

These and other, further and more specific objects and advantages of the invention will be apparent from the following detailed description thereof, taken in conjunction with the drawings, in which:

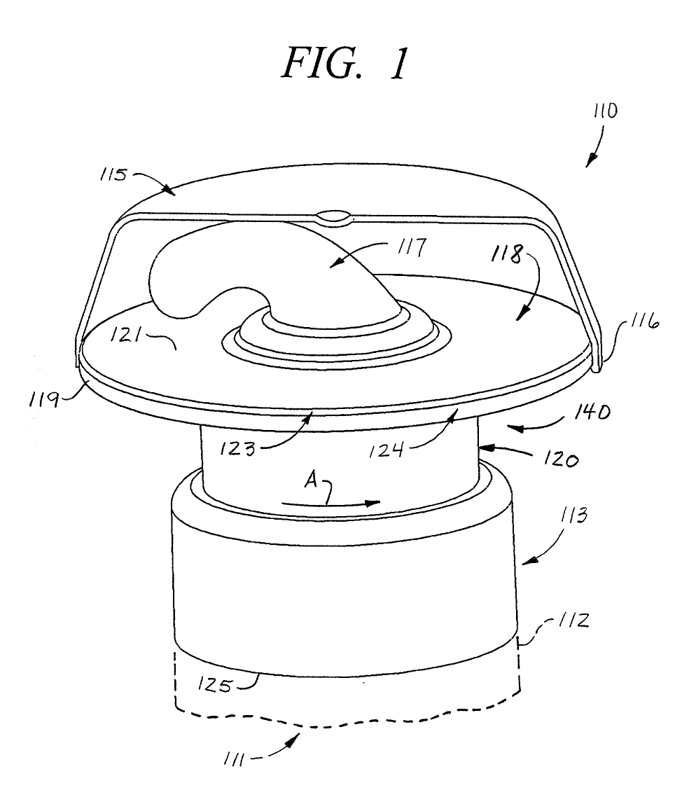

FIG. 1 is a perspective view illustrating a pre-sterilized, sealed, disposable, pre-packaged, self-contained, reusable safety baby bottle constructed in accordance with the principles of the invention;

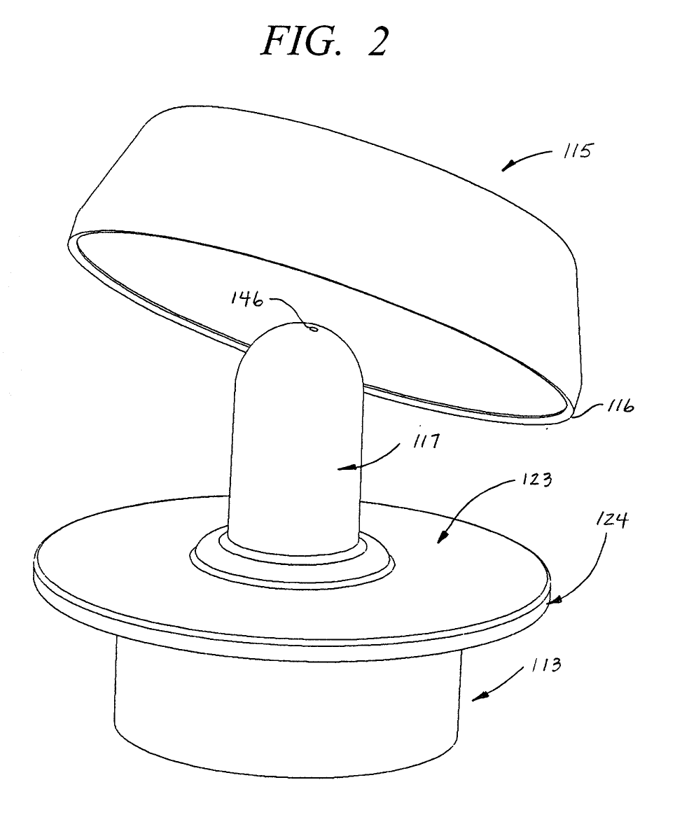

FIG. 2 is a perspective view illustrating the mode of operation of the baby bottle of FIG. 1;

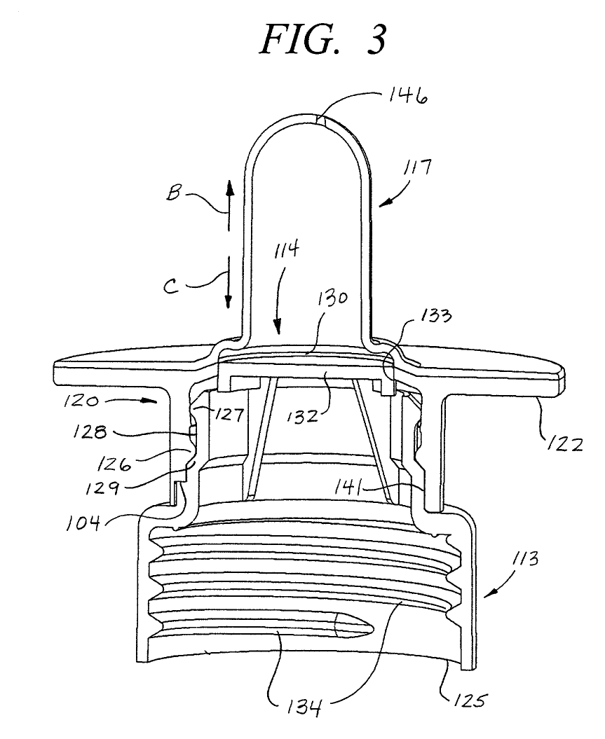

FIG. 3 is a perspective view further illustrating the mode of operation of the baby bottle of FIG. 1;

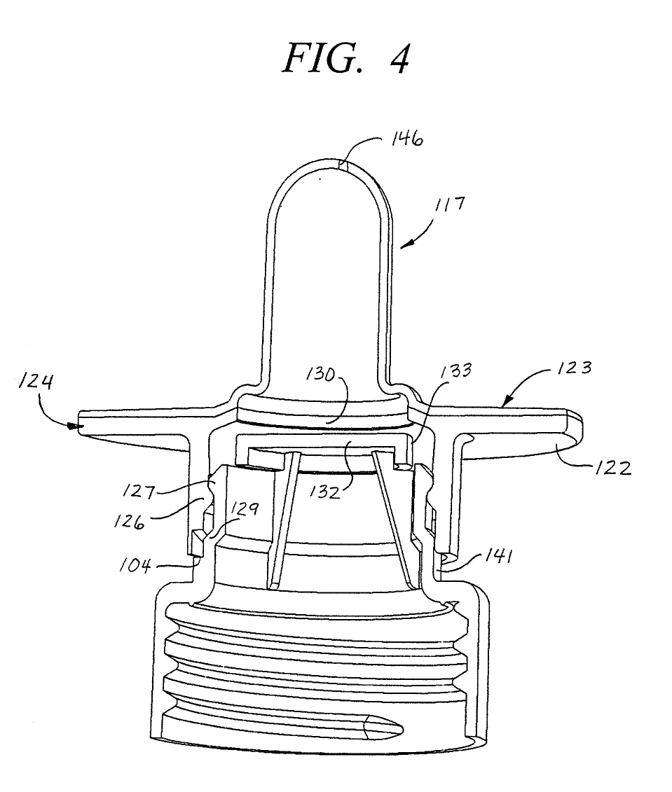

FIG. 4 is a perspective view still further illustrating the mode of operation of the baby bottle of FIG. 1;

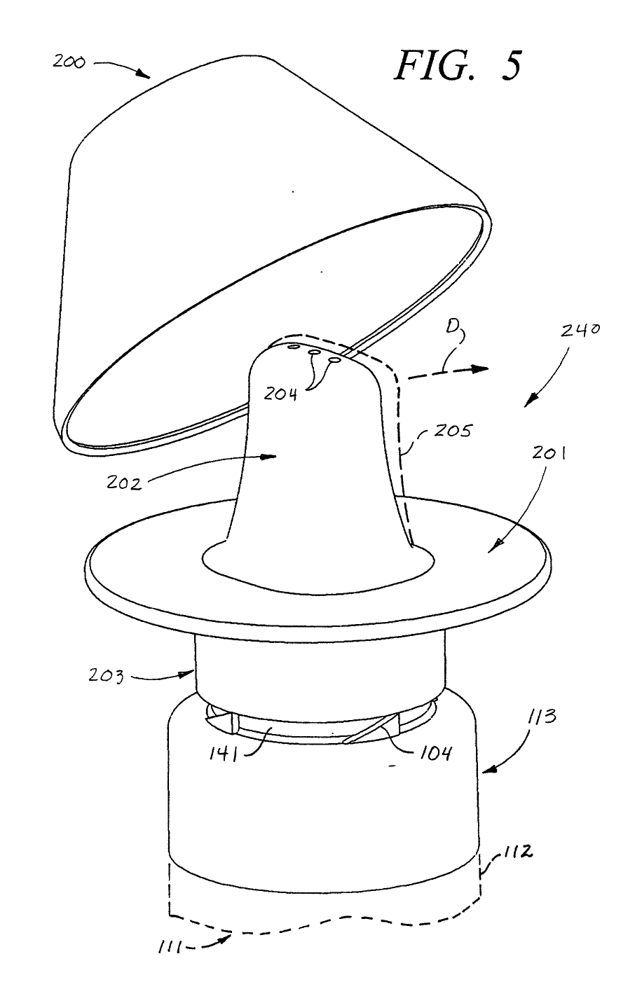

FIG. 5 is a perspective view illustrating another embodiment of the pre-sterilized, sealed, disposable, pre-packaged, self-contained, reusable safety bottle of the invention;

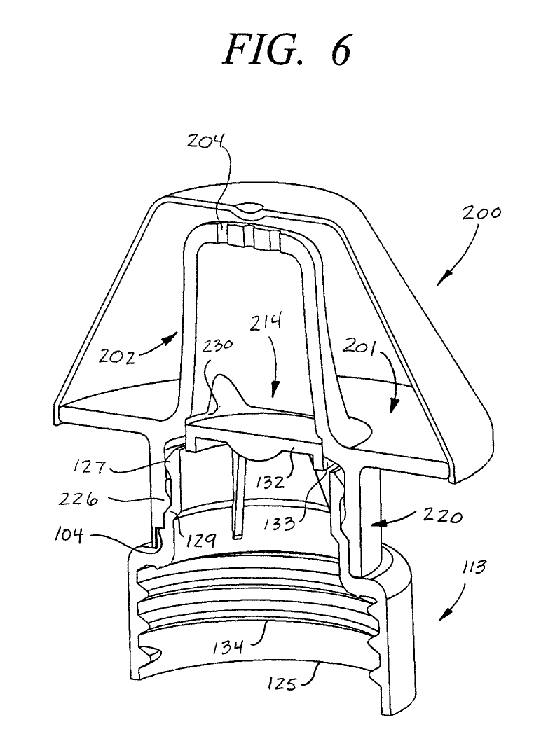

FIG. 6 is a perspective view illustrating the mode of operation of the baby bottle of FIG. 5; and,

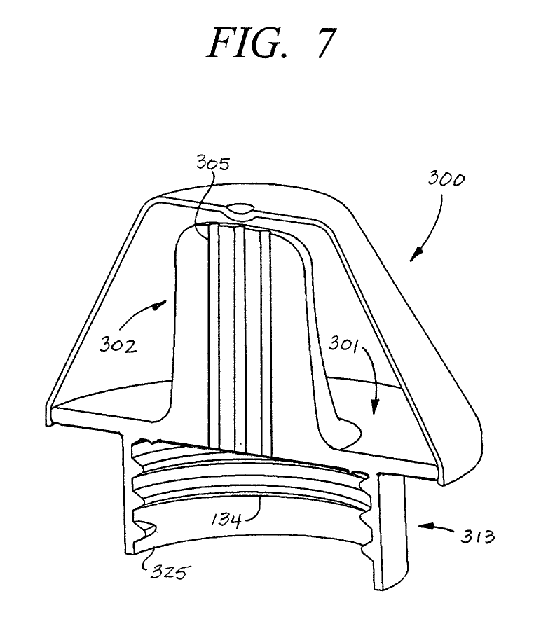

FIG. 7 is a perspective view illustrating still another embodiment of the pre-sterilized, sealed, disposable, pre-packaged, self-contained, reusable safety bottle of the invention.

Briefly, in accordance with the invention, I provide an improved method for a parent to safely administer formula to a baby. The method includes the step of providing a pre-packaged, pre-sterilized, self-contained, disposable safety bottle by sterilizing a container; sterilizing formula; charging the sterilized container with the sterilized formula; sealing the container; and, removably mounting a nipple assembly on the container. The nipple assembly includes a base, a nipple, and a removable cover extending over the nipple. The method also includes the steps of delivering the safety bottle to the parent; removing the nipple assembly from the container; unsealing the container; remounting the nipple assembly on the container; removing the cover; administering the formula to the baby; and, discarding the safety bottle.

In another embodiment of the invention, I provide an improved method for a parent to safely administer formula to a baby. The method includes the step of providing a pre-packaged, pre-sterilized, self-contained, disposable safety bottle by sterilizing a container; sterilizing formula; charging the sterilized container with the sterilized formula; and, removably mounting a nipple assembly on the container. The nipple assembly includes a base; a nipple; a removable cover extending over the nipple, and a valve. The valve is movable between at least two operative positions, a first sealed operative position preventing formula from flowing from the container through the nipple, and a second open operative position permitting formula from the container to flow through the nipple. The nipple assembly is mounted on the container with the valve in the first operative position to seal the formula in the container. The method also includes the steps of delivering said safety bottle to the parent; unsealing the container by moving the valve from the first operative position to the second operative position; removing the cover; administering the formula to the baby; and, discarding the safety bottle.

Turning now to the drawings, which depict the presently preferred embodiments of the invention for the purpose of illustrating the practice thereof and not by way of limitation of the scope of the invention, and in which like reference characters refer to corresponding elements throughout the several views, FIGS. 1 to 4 illustrate a baby bottle 110 constructed in accordance with the invention and generally indicated by reference character 110 and including a bottle 111 with top or neck 112, base 113 that mounts on neck 112, displaceable top 140, and removable cap 115 covering nipple 117. Top 140 includes sleeve 120, circular collar 118, and pliable nipple 117 mounted on collar 118. Collar 118 presently, although not necessarily, has a laminate construction comprising cylindrically shaped layers 123, 124.

Base 113 is shaped and dimensioned to be sealingly mounted on neck 112.

In the embodiment of base 113 illustrated in the drawings, neck 112 is externally threaded and base 113 is internally threaded. Base 113 includes lower circular lip 125 and continuous helical internal thread 134 shaped and dimensioned to be turned onto the external threads (not visible) on the top 112 of container 111. The shape and dimension of base 113 can vary as desired as long as base 113 can be mounted on a liquid container 111.

Valve 114 includes portions integrally formed with base 113 and top 140. Valve 114 includes a hollow upstanding neck depending upwardly and including generally cylindrical outer surface 141 (FIGS. 3 and 4) and a canted track 104 formed in surface 141. Valve 140 also includes outwardly projecting circular raised member 127, includes circular shoulder 129, includes circular groove 128 extending between member 127 and shoulder 129, and includes circular panel 132 (FIGS. 8, 9).

Panel 132 includes circular peripheral sealing edge 133. Circular sealing edge 130 is formed in top 140. Top 140 moves up (in the direction of arrow B) and down (in the direction of arrow C) on base 113 in the manner of “pop-tops” commonly found on plastic water bottles.

Rib 126 is formed on the interior of sleeve 120. When member 140 moves up and down in the direction of arrows B and C, circular rib 126 slides upwardly and downwardly in groove 128. Groove 128 extends between member 127 and shoulder 129.

A pin (not visible) or other member is connected to the inside of sleeve 120. The pin extends inwardly from sleeve 120, and—when top 140 is rotated in the direction of arrow A—slides upwardly along track 104 to upwardly displace top 140 in the direction of arrow B from the position shown in FIG. 3 to the position shown in FIG. 4. When top 140 is in the position shown in FIG. 4, and top 140 is turned in a direction opposite that of arrow A, then the pin slides downwardly along track 104 and top 140 moves from the position shown in FIG. 4 to the position shown in FIG. 3. When member 140 moves between the positions shown in FIGS. 3 and 4, rib 126 rotates in and slides upwardly and downwardly in groove 128 between member 127 and shoulder 129. As would be appreciated by those of skill in the art, other conventional pop-top assemblies are available in which the top 140 pulls straight up or down and need not be rotated.

When top 140 is in the position illustrated in FIG. 3 sealing edge 133 of circular panel 132 is sealingly adjacent inner circular edge 130. Edge 133 cooperates with edge 130 to prevent or substantially prevent liquid from flowing out of container 111 under gravity intermediate edges 130-133 when container 111 and the valve 114 are inverted.

When top 140 is in the position illustrated in FIG. 4, sealing edge 130 is upwardly displaced and spaced away from edge 133 such that liquid can flow out of container 111 under gravity intermediate edges 130 and 133 when container 111 and valve 114 are inverted.

As would be appreciated by those of skill in the art, valve 114 can be constructed in any desired manner as long as the valve 114 has at least a pair of operative positions, a first operative position in which liquid is prevented from flowing out of container 111 and a second operative position in which liquid is permitted to flow out of container 111. Another example of a valve which can be moved between two operative positions is the well known “sport cap” found on plastic bottles of water. The sport cap includes an outer sleeve which can be pulled up away from the top of the bottle to open the cap so water will flow out of the bottle. The sleeve is pushed toward the bottle to close the cap so water will not flow out of the bottle. The sport cap valve mechanism and construction can be utilized in place of the open-close mechanism illustrated in the drawings.

As noted, substantially rigid laminate collar 118 includes a circular upper polymer layer 123 and a lower polymer layer 124. Layer 123 and nipple 117 are each presently formed from the same polymer material and are integrally formed together. Layer 123 includes upper planar surface 121 circumscribing nipple 117. Layer 124 is integrally formed with sleeve 120 and includes lower planar surface 122 circumscribing sleeve 120. Collar 118 functions to prevent a baby's mouth from reaching and extending over sleeve 120. Instead, the baby's lips contact collar 118. Collar 118 presently preferably has a diameter or width of at least thirty-eight millimeters, preferably forty millimeters, and most preferably at least forty-five millimeters.

In the embodiment of the invention illustrated in FIGS. 5 and 6, top 140 is replaced with a top 240 including a nipple 202 that has at least one opening 204 formed therethrough. Nipple 202 is mounted in a cylindrical collar 201 attached to a sleeve 203. Top 240 is mounted on the same base 113 as top 140. The circular sealing surface 230 formed on the interior of top 240 has a shape and dimension equivalent to that of sealing surface 130 of top 140. The shape and dimension of rib 226, sleeve 220, and valve 214 are equivalent to that of rib 126, sleeve 120, and valve 214, respectively. Top 240 moves up and down in the same manner as top 140, i.e., moves between a first position where valve 214 is closed and a second position where valve 214 is open.

The embodiment of the invention illustrated in FIG. 7 is similar to the embodiment shown in FIGS. 5 and 6, except that the apertures 305 formed in nipple 302 are always open. Cylindrical collar 301 is permanently secured to a base 313. Base 313 includes internal threads 134 which turn onto the externally threaded neck 112 of a bottle 111 or other container. Cover 300 snaps onto collar 301 to protect nipple 302. When the embodiment of the invention illustrated in FIG. 7 is mounted on the neck 112 of a bottle 111, fluid can be continuously sucked out through apertures 305 when bottle 111 is inverted. Nipples 302 is preferably made out of a polymer that permits a baby to deform elastically and displaced nipple 302 about one-half inch to either side in the manner indicated by dashed lines 205 and arrow D in FIG. 5. Nipple 302 is also, however, substantially solid such that a baby or young child can not readily bite or bend nipple 302 to constrict apertures 305 and prevent fluid from flowing through the apertures. In contrast, nipples 202 and 117 typically can be bitten or squeezed by the mouth of a child to prevent formula, water, juice, or some other fluid in bottle 111 from flowing out through the opening(s) 146, 204 in the nipple.

In use, a baby bottle or other container 111 is provided. The container can be fabricated from plastic, glass, or any other desired material. The container usually, but not necessarily, will be discarded after liquid in the container is consumed by a baby or child. The container is sterilized.

Milk, soy milk, or some other formula is provided. The formula is sterilized and put into the container 111. Water, juice, or some other fluid can also be put in container 111 instead of or in conjunction with formula. Water, juice, and fluids other than formula may not require sterilization, but may only require some preliminary purification steps, or pasteurization steps, etc.

The sterilized formula is put into the sterilized container. The container is sealed. One preferred way of sealing the container is by placing a circular seal over the top edge of the neck of the bottle. The seal can include a layer of aluminum or other metal laminated on paper. The seal can be constructed in any desired manner. The normal function of the seal is to prevent contaminants from entering the bottle 111 and to prevent liquid from escaping from the bottle. The seal can be peeled off the bottle when it is desired to use the bottle to feed a baby. Another way of sealing the container is to place one of the tops of FIGS. 1 to 6 on the neck 112 with the valve 114, 214 in the closed position.

After the baby bottle is sealed, a top is mounted on the neck 112. The top includes a nipple 117, 202, 302 and includes a cover 115, 200, 300 that snaps onto the top 140, 240 to cover and protect the nipple 117, 202, 302. The nipple 117, 202, 302 and cover 115, 200, 300 preferably are sterilized so that after cover 115, 200, 300 is applied the sterile nature of the nipple is preserved until cover 115, 200, 300 is removed when bottle 111 is being used to feed a baby.

When the bottle 111, base 113, top 140, and cover 115 are assembled, a pre-packaged, pre-sterilized, self-contained, disposable safety bottle is produced. The safety bottle does not require sterilization prior to use. The safety bottle need not be charged with formula or other liquid prior to use. Use of the safety bottle does not require clean up of various utensils which are commonly used to produce formula. A supply of baby bottles is not necessary. A supply of nipples is not necessary. A supply of formula is not necessary. Sterilization equipment is not necessary. Bottles, nipples, and equipment need not be stored in sealed area free from insects and chemical contaminants.

The safety baby bottles are delivered to parents by distributing the bottles to grocery stores and other outlets and by the outlets selling the safety baby bottles to parents. The parent transports purchased safety bottles to home or some other desired location. The parent unthreads base 113 from the neck 112, and peels the seal off the top of the neck 112 of the bottle 111. The base 113 is sealingly threaded back onto neck 112. Protective cover 115 is removed. If necessary, collar 118 is pulled or turned to displace top 140 upwardly in the direction of arrow B to open valve 114. Nipple 117 is placed directly in the mouth of a baby or young child. After the formula or other liquid in the bottle is administered to the baby, the bottle 11 is discarded. If desired, bottle 111 can be resealed by downwardly displacing top 140 in the direction of arrow C to close valve 114. This procedure for administering formula or other liquids to a baby makes it much less likely that the formula or other liquid in bottle 111 will be contaminated. The procedure also greatly simplifies traveling with a baby, because the disposable safety bottle of the invention eliminates many of the afore-mentioned problems associated with feeding a baby “on the go”.

In one preferred embodiment of the invention, the base 113 is sized to fit on the 28 millimeter diameter externally threaded neck of a conventional water bottle. Such conventional bottles are readily available and widely utilized in grocery stores and other retail outlets and come in various convenient sizes.

Nipple 117 or any other desired portion of the assembly 110 can be impregnated with a chemical composition which produces a scent that smells like a cherry, blueberry, or other desired fragrance. Such a fragrance can give a person drinking a fluid through assembly 110 the impression that the fluid includes cherry, blueberry, or another food associated with the fragrance. One use of such chemical compositions is set forth in U.S. Pat. No. 6,045,833.

In FIG. 5 (and FIG. 7) the openings 204 formed in nipple 202 preferably, but not necessarily, permit a cumulative volume of fluid to flow out of openings 204 (when the valve 214 is open) which is less than that which flows out of a conventional pop-top that is currently found on water bottles and is used by adults. The combined volume of fluid which exits all of the openings 204 when the valve 214 is open is presently preferably, but not necessarily, equivalent to the volume of liquid which flows out of the spout on a tippee-cup top. Tippee-cup spouts typically are the next step in teaching a young child to drink. The child graduates from a baby nipple to a tippee cup spout, and then to drinking out of a glass or cup which does not have a cover. The shape and dimension of nipple 202 can vary as desired, but nipple 202 is presently shaped to fit in the mouth of a young child one to four years old. Collar 201 is shaped and dimensioned to prevent a child from getting his mouth completely over the entire collar 201.

As earlier noted, the baby bottles illustrated in FIGS. 1 to 7 are preferably each utilized by sterilizing or otherwise disinfecting a bottle or other container, by charging the bottle with sterile fluid, by sterilizing or otherwise disinfecting the top, by mounting the top on the bottle that was charged with fluid to seal the bottle (or by otherwise sealing the top of the bottle with a removable or puncturable seal before mounting the bottle top on the bottle), by covering the top with a sterilized or disinfected cover or plastic coating to protect the top from contamination, by selling the pre-sterilized, pre-sealed safety baby bottle to a consumer, by removing the seal protecting the baby bottle, by (if necessary) opening a valve 114 in the top 140 of the safety bottle, by having a young child or infant drink fluid from the bottle, and by discarding the safety bottle. This procedure greatly simplifies and improves on the safety of the age-old procedures for feeding a baby.

Having described my invention in such terms as to enable those of skill in the art to understand and use it, and having described the presently preferred embodiments and best mode thereof,

I claim:

1. A method for a parent to safely administer formula to a baby, including the steps of
(a) providing a pre-packaged, pre-sterilized, self-contained, disposable safety bottle by
(i) sterilizing a container,
(ii) sterilizing formula,
(iii) charging said sterilized container with said sterilized formula,
(iv) sealing said container,
(v) removably mounting a nipple assembly on said container, said nipple assembly including a base, a nipple, and a removably cover extending over said nipple;
(b) delivering said safety bottle to the parent;
(c) removing said nipple assembly from said container;
(d) unsealing said container;
(e) remounting said nipple assembly on said container;
(f) removing said cover;
(g) administering the formula to the baby; and,
(h) discarding said safety bottle.

2. A method for a parent to safely administer formula to a baby, including the steps of
(a) providing a pre-packaged, pre-sterilized, self-contained, disposable safety bottle by
(i) sterilizing a container,
(ii) sterilizing formula,
(iii) charging said sterilized container with said sterilized formula,
(iv) removably mounting a nipple assembly on said container, said nipple assembly including a base, a nipple, a removable cover extending over said nipple, and valve means movable between at least two operative positions,
a first sealed operative position preventing formula from flowing from said container through said nipple, and
a second open operative position permitting formula from the container to flow through said nipple,
said nipple assembly being mounted on said container with said valve means in said first operative position to seal said formula in said container;
(b) delivering said safety bottle to the parent;
(c) unsealing said container by moving said valve means from said first operative position to said second operative position;
(d) removing said cover;
(e) administering the formula to the baby; and,
(f) discarding said safety bottle.
